(19) United States
(12) Patent Application Publication
(10) Pub. No.: US 2012/0042894 A1
(43) Pub. Date: Feb. 23, 2012

(54) COMPOSITION COMPRISING THE COMBINATION OF MADECASSOSIDE, OF AN ARGININE AND OF POLYSORBATE

(75) Inventors: Isabelle Jacquier, Chevilly Larue (FR); Jean-Francois Nadaud, Issy-les-Moulineaux (FR)

(73) Assignee: L'OREAL, PARIS (FR)

(21) Appl. No.: 13/257,608

(22) PCT Filed: Mar. 10, 2010
(86) PCT No.: PCT/EP2010/053030

§ 371 Date: Oct. 25, 2011

Related U.S. Application Data
(60) Provisional application No. 61/163,529, filed on Mar. 26, 2009.
(60) Provisional application No. 61/250,949, filed on Oct. 13, 2009.

(30) Foreign Application Priority Data
Mar. 20, 2009 (FR) 0951804

(51) Int. Cl. A45D 40/26; A45D 34/04; A61Q 7/00; A61Q 1/10; A61K 8/60; A61Q 5/00

(52) U.S. Cl. 132/202; 514/33; 424/70.1; 132/218

(57) ABSTRACT

The invention relates to a composition for caring for human keratinous fibres comprising, in a physiologically acceptable medium: a) at least one compound chosen from arginines, their organic or inorganic acid salts and their derivatives; b) at least one compound of the formula (I) in which R₁=H or —CH₃; R₂=H or —CH₃; R₃=—CH₃, R₁ and R₂ not simultaneously being H, or else a Centella asiatica extract comprising a compound or a mixture of compounds of formula (I); and optionally c) at least one compound of formula (II) in which w+x+y+z has a mean value of 4.

A subject-matter of the invention is a cosmetic composition for coating human keratinous fibres, intended in particular to reduce or slow down their loss and/or to induce and/or stimulate their growth and/or increase their density, comprising, in a physiologically acceptable medium:

a) at least one compound chosen from arginines, their organic or inorganic acid salts and their derivatives;
b) at least one compound of formula (I) or else a Centella asiatica extract comprising them; and
c) at least one compound of formula (II).

It additionally relates to a cosmetic treatment method intended to induce and/or stimulate the growth of human keratinous fibres and/or to slow down their loss and/or to increase their density.

The treatment method according to the invention can be carried out using an applicator capable of being driven with a vibratory movement, alone or in combination with a composition comprising i) at least one compound chosen from arginines, their organic or inorganic acid salts and their derivatives; ii) at least one compound of formula (I) or else a Centella asiatica extract comprising them; and, optionally, iii) at least one compound of formula (II).

Beneficial effects on keratinous fibres have also been observed on applying the composition targeted above with a conventional applicator, that is to say a nonvibrating applicator.

The human keratinous fibres to which the invention applies are in particular the hair, eyebrows and eyelashes. More especially, the invention applies to human eyelashes and eyebrows.

In man, the growth of the hair, eyebrows and eyelashes and their replacement are mainly determined by the activity of the hair follicles and of their dermoepidermal environment. Their activity is cyclical and essentially comprises three phases, namely the anagen phase, the catagen phase and the telogen phase.

A search has been underway for many years, in particular in the cosmetics industry, for compositions which make it possible to induce or stimulate the growth of the hair and/or eyelashes and/or to reduce or delay their loss.

The Applicant Company has discovered, surprisingly, during its research studies, that the combination a) of an arginine and/or of one of its organic or inorganic acid salts and/or one of its derivatives, with b) a compound or a mixture of compounds of formula (I) which will be defined later or else an extract of Centella asiatica comprising the said compound or compounds of formula (I), and with c) a compound of formula (II), results in a substantially greater effectiveness in combating hair loss, with respect to each of the active principles used alone.

A subject-matter of the invention is a cosmetic composition for coating human keratinous fibres comprising, in a physiologically acceptable medium:

a) at least one compound chosen from arginines, their organic or inorganic acid salts and their derivatives;
b) at least one compound of formula (I) or else a Centella asiatica extract comprising a compound or a mixture of compounds of formula (I); and
c) at least one compound of formula (II).

The invention also relates to the cosmetic use of the combination:

a) of at least one compound chosen from arginines, their organic or inorganic acid salts and their derivatives;
b) of at least one compound or a mixture of compounds of formula (I) or else a Centella asiatica extract comprising a compound or a mixture of compounds of formula (I), and
c) of at least one compound of formula (II), in a cosmetic composition for coating human keratinous fibres, with the aim of inducing and/or stimulating the growth of the said fibres and/or slowing down their loss and/or increasing their density.

Increasing the density of human keratinous fibres and in particular the density of the eyelashes or eyebrows is understood to mean increasing the number of keratinous fibres, in particular eyelash or eyebrow fibres, per cm² of skin, such as the eyelids. Thus, this composition makes it possible to keep the eyelashes or eyebrows in a good condition.

Another subject-matter of the present invention is a method for the cosmetic treatment of human keratinous fibres intended to induce and/or stimulate the growth of the said fibres, in particular the eyelashes or eyebrows, and/or to slow down their loss and/or to increase their density, the method consisting in applying, to these fibres, a cosmetic composition as defined above.

Another subject-matter of the present invention is a method for the cosmetic treatment of human keratinous fibres intended to induce and/or stimulate the growth of the said fibres, in particular the eyelashes or eyebrows, and/or to slow down their loss and/or to increase their density, the method consisting in causing the fibres to engage with an applicator, in particular a brush or a comb, capable of being driven with a vibratory movement.

Another subject-matter of the present invention is a method for the cosmetic treatment of human keratinous fibres intended to induce and/or stimulate the growth of the said fibres, in particular the eyelashes or eyebrows, and/or to slow down their loss and/or to increase their density, the method consisting in applying, to the fibres, a composition comprising i) at least one compound chosen from arginines, their organic or inorganic acid salts and their derivatives; ii) at least one compound of formula (I) or else a Centella asiatica extract comprising a compound or a mixture of compounds of formula (I); and, optionally, iii) at least one compound of formula (II), the said application being combined with causing the said fibres to engage, simultaneously with the application or separately, with a device, in particular a brush or a comb, capable of being driven with a vibratory movement.

Advantageously, the composition is applied using a vibrating applicator.

This treatment method certainly exhibits the characteristics of a cosmetic method in so far as it makes it possible to improve the aesthetics of human keratinous fibres and in particular of the eyelashes by giving them greater vigour and an improved appearance. In addition, it can be used daily for several months.

Finally, a subject-matter of the present invention is a combination for making up or caring for the eyelashes or eyebrows, comprising:
at least one composition according to the invention; and
at least one applicator for the composition, the said applicator comprising means which make it possible to smooth and/or separate the eyelashes or eyebrows, in particular in the form of teeth, bristles or other raised patterns.

Advantageously, the applicator is capable of being driven with a vibratory movement.

The compositions in accordance with the invention comprise, as first active principle, an arginine and/or one of its salts and/or one of its derivatives.

The various forms of arginine are generally D-arginine, D,L-arginine and L-arginine. Mention may be made, among the organic or inorganic acid salts of arginine, for example, of the hydrochlorides, glutamates, butyrates and glycolates.

The term “arginine derivatives” is understood to mean, within the meaning of the invention, arginines carrying substitutions either on the carboxyl group of the said arginines or on the amino group carried by the carbon in the α position with regard to the carboxyl group or optionally on both these groups.

Mention may be made, among L-arginine derivatives, of C₁-C₄ alkyl esters of L-arginine, the alkyl being chosen, for example, from methyl, ethyl, propyl, isopropyl, butyl or isobutyl.

Mention may also be made, among L-arginine derivatives, of L-arginine oligomers (known as polyarginine), such as those described in Application EP 1 060 739 and in Application WO 03/072039.

Arginine oligomers preferred according to the invention comprise from 7 to 15 L-arginine units, for example 7, 9, 11, 13 or 15 L-arginine units.

Use will more particularly be made of L-arginine.

The arginine or arginines and/or their salts and/or their derivatives in accordance with the invention will be present in the compositions in amounts preferably ranging from 0.01% to 5% of the total weight of the composition and more preferably in an amount representing from 0.02% to 2% of the total weight of the composition and more preferably still from 0.03% to 1%.

The compound or compounds of formula (I) in accordance with the invention correspond to the following formula:

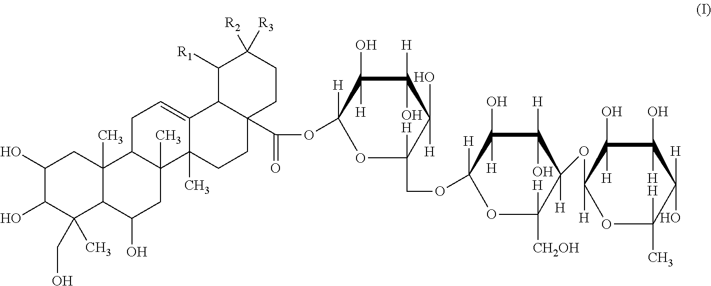

in which R₁=H or —CH₃; R₂=H or —CH₃; R₃=—CH₃, R₁ and R₂ not simultaneously being H.

The preferred compounds of formula (I) are madecassoside (compound of formula (I) with R₁=R₃=—CH₃ and R₂=H) and terminoloside (compound of formula (I) with R₁═H and R₂=R₃=—CH₃).

These compounds of formula (I) and in particular their mixtures can especially be extracted from Centella asiatica according to the process described in Application WO 2004/062678.

According to a preferred form of the invention, use is made of a mixture of madecassoside and of terminoloside. More preferably, the mixture of madecassoside and of terminoloside has a madecassoside content ranging from 30 to 70% by weight, with respect to the total weight of the mixture, and more particularly ranging from 45 to 55% by weight and more particularly of 50% by weight.

According to a particularly preferred form of the invention, use will be made of a Centella asiatica extract comprising more than 95% by weight of the madecassoside/terminoloside mixture, with respect to the weight of the extract.

A Centella asiatica extract comprising more than 95% by weight of madecassoside/terminoloside mixture (50/50% by weight) is sold in particular under the trade name Madecassoside by Bayer. This mixture has the CTFA name Madecassoside.

The compound or the mixture of compounds of formula (I) described above can be present in the composition for topical application in an amount ranging, for example, from 0.01 to 5% by weight and preferably from 0.1 to 2% by weight, with respect to the total weight of the composition.

The compound or compounds of formula (II) used in accordance with some embodiments of the invention correspond to the following formula:

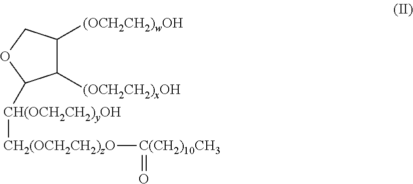

in which w+x+y+z has a mean value of 4.

According to a preferred form of the invention, use is made of the mixture of compounds of formula (II) sold by Croda under the reference Tween 21 (INCI name: Polysorbate-21).

The compound or the mixture of compounds of formula (II) described above can be present in the composition for topical application in an amount ranging, for example, from 0.01 to 5% by weight and preferably from 0.1 to 2% by weight, with respect to the total weight of the composition.

Preferably, the composition according to the invention comprises, in addition to the compounds a), b) and optionally c), at least one compound d) chosen from citric acid and its salts.

Mention may be made, among the salts of citric acid, of the salts of citric acid and of an alkali metal or alkaline earth metal. Such salts are in particular potassium citrate or sodium citrate.

Preferably, the compound d) is citric acid.

### Physiologically Acceptable Medium

The compositions used according to the invention can constitute in particular cosmetic and dermatological compositions. They comprise, for such an application, a physiologically acceptable medium. “Physiologically acceptable medium” is understood here to mean a medium compatible with the skin and optionally with the lips, scalp, eyelashes, eyebrows, eyes and/or the hair.

The composition according to the invention can be applied topically to human keratinous fibres, in particular to the eyelashes or eyebrows.

This composition can be provided in any known formulation form suited to the method of use.

For application to the eyelashes or eyebrows, the composition to which the invention applies can be provided in particular in the form of a mascara, of a serum, of an eyeliner or of a gel, which may or may not be pigmented, to be applied with the brush to the eyelashes or eyebrows.

For topical application to the skin, including the scalp, the composition can have the form of an aqueous, alcoholic, aqueous/alcoholic or oily solution or suspension, of an emulsion or dispersion with a more or less fluid consistency and in particular a liquid or semiliquid consistency, obtained by dispersion of a fatty phase in an aqueous phase (O/W) or vice versa (W/O), of a solid (O/W) or (W/O) emulsion or dispersion, of a more or less fluid or solid aqueous, aqueous/alcoholic or oily gel, of a loose or compact powder, to be used as is or to be incorporated in a physiologically acceptable medium, or also of microcapsules or microparticles, or of vesicular dispersions of ionic and/or nonionic type.

The amounts of the various constituents of the physiological medium of the composition according to the invention are those generally used in the fields under consideration. In addition, these compositions are prepared according to the usual methods.

For an application of mascara type, the composition is preferably provided in the form of a wax-in-water emulsion or of a wax-in-oil dispersion, which may or may not be pigmented.

The composition according to the invention preferably comprises at least one compound chosen from waxes, film-forming polymers, gelling polymers, surfactants, oils, colouring materials, fillers, fibres and their mixtures.

Waxes

The term “wax” is understood to mean, within the meaning of the present invention, a lipophilic compound which is solid at ambient temperature (25° C.), which exhibits a reversible solid/liquid change in state and which has a melting point of greater than or equal to 30° C. which can range up to 120° C.

The melting point of the wax can be measured using a differential scanning calorimeter (DSC), for example the calorimeter sold under the name DSC 30 by Metler.

The waxes can be hydrocarbon, fluorinated and/or silicone waxes and can be of vegetable, mineral, animal and/or synthetic origin. In particular, the waxes exhibit a melting point of greater than 25° C. and better still of greater than 45° C.

The wax is present in a content at least equal to 5% by weight. Preferably, it is present in a content ranging from 5 to 50% by weight, with respect to the total weight of the composition, better still from 10 to 40% by weight and even better still from 15 to 35% by weight.

Use may in particular be made of hydrocarbon waxes, such as beeswax, lanolin wax and Chinese insect waxes; rice wax, carnauba wax, candelilla wax, ouricury wax, esparto wax, cork fibre wax, sugarcane wax, Japan wax and sumac wax; montan wax, microcrystalline waxes, paraffin waxes and ozokerite; polyethylene waxes, waxes obtained by the Fischer-Tropsch synthesis and waxy copolymers, and also their esters.

Mention may also be made of the waxes obtained by catalytic hydrogenation of animal or vegetable oils having linear or branched C₈-C₃₂ fatty chains.

Mention may in particular be made, among these, of hydrogenated jojoba oil, hydrogenated sunflower oil, hydrogenated castor oil, hydrogenated coconut oil, hydrogenated lanolin oil, di(1,1,1-trimethylolpropane) tetrastearate, sold under the name “Hest 2T-4S” by Heterene, or di(1,1,1-trimethylolpropane) tetrabehenate, sold under the name “Hest 2T-4B” by Heterene.

Mention may also be made of silicone waxes, such as alkyl or alkoxy dimethicones having from 16 to 45 carbon atoms, or fluorinated waxes.

Use may also be made of the wax obtained by hydrogenation of olive oil esterified with stearyl alcohol sold under the name “Phytowax Olive 18 L 57” or else of the waxes obtained by hydrogenation of castor oil esterified with cetyl alcohol sold under the names “Phytowax castor 16L64” and “Phytowax castor 22L73” by Sophim. Such waxes are described in Application FR-A-2 792 190.

According to a specific embodiment, the compositions in accordance with the invention can comprise at least one wax known as “tacky wax”, that is to say having a tack of greater than or equal to 0.7 N.s and a hardness of less than or equal to 3.5 MPa.

The use of a tacky wax can in particular make it possible to obtain a cosmetic composition which is easily applied to the eyelashes, which has good attachment to the eyelashes and which results in the formation of a smooth, homogeneous and thickening make-up.

The tacky wax used can in particular have a tack ranging from 0.7 N.s to 30 N.s, in particular of greater than or equal to 1 N.s, in particular ranging from 1 N.s to 20 N.s, especially of greater than or equal to 2 N.s, in particular ranging from 2 N.s to 10N.s, and especially ranging from 2 N.s to 5 N.s.

The tack of the wax is determined by the measurement of the change in the force (compressive force or stretching force) as a function of the time at 20° C. using the texture analyser sold under the name “TA-TX2i®” by Rheo, equipped with a spindle made of acrylic polymer in the shape of a cone forming an angle of 45°.

The measurement protocol is as follows:

The wax is melted at a temperature equal to the melting point of the wax +10° C. The molten wax is cast in a receptacle with a diameter of 25 mm and a depth of 20 mm. The wax is recrystallized at ambient temperature (25° C.) for 24 hours, so that the surface of the wax is flat and smooth, and then the wax is stored at 20° C. for at least 1 hour before measuring the tack.

The spindle of the texture analyser is displaced at the rate of 0.5 mm/s and then penetrates the wax to a penetration depth of 2 mm. When the spindle has penetrated the wax to a depth of 2 mm, the spindle is held stationary for 1 second (corresponding to the relaxation time) and is then withdrawn at the rate of 0.5 mm/s.

During the relaxation time, the force (compressive force) strongly decreases until it becomes zero and then, during the withdrawal of the spindle, the force (stretching force) becomes negative to subsequently again increase towards the value of 0. The tack corresponds to the integral of the curve of the force as a function of the time for the part of the curve corresponding to the negative values of the force (stretching force). The value of the tack is expressed in N.s.

The tacky wax which can be used generally has a hardness of less than or equal to 3.5 MPa, in particular ranging from 0.01 MPa to 3.5 MPa, especially ranging from 0.05 MPa to 3 MPa, indeed even ranging from 0.1 MPa to 2.5 MPa.

The hardness is measured according to the protocol described above.

Use may be made, as tacky wax, of a C₂₀-C₄₀ alkyl (hydroxystearyloxy)stearate (the alkyl group comprising from 20 to 40 carbon atoms), alone or as a mixture, in particular a C₂₀-C₄₀ alkyl 12-(12′-hydroxystearyloxy)stearate, of formula (II):

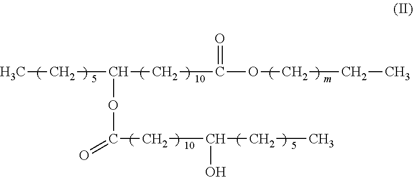

in which m is an integer ranging from 18 to 38, or a mixture of compounds of formula (II).

Such a wax is sold in particular under the names “Kester Wax K 82 P®” and “Kester Wax K 80 P®” by Koster Keunen.

The abovementioned waxes generally exhibit a starting melting point of less than 45° C.

Use may also be made of the microcrystalline wax sold under the reference SP18 by Strahl and Pitsch, which exhibits a hardness of approximately 0.46 MPa and a tack value of approximately 1 N.s.

The wax or waxes can be present in the form of an aqueous wax microdispersion. The term “aqueous wax microdispersion” is understood to mean an aqueous dispersion of wax particles in which the size of the said wax particles is less than or equal to approximately 1 μm.

Wax microdispersions are stable dispersions of colloidal wax particles and are described in particular in “Microemulsions Theory and Practice”, edited by L. M. Prince, Academic Press (1977), pages 21-32.

In particular, these wax microdispersions can be obtained by melting the wax in the presence of a surfactant and optionally of a portion of the water and then gradually adding hot water with stirring. The intermediate formation of an emulsion of the water-in-oil type, followed by phase inversion, with a microemulsion of oil-in-water type finally being obtained, is observed. On cooling, a stable microdispersion of solid colloidal wax particles is obtained.

The wax microdispersions can also be obtained by stirring the mixture of wax, of surfactant and of water using stirring means, such as ultrasound, a high pressure homogenizer or turbine mixers.

The particles of the wax microdispersion preferably have mean sizes of less than 1 μm (in particular ranging from 0.02 μm to 0.99 μm), preferably of less than 0.5 μm (in particular ranging from 0.06 μm to 0.5 μm).

These particles are composed essentially of a wax or of a mixture of waxes. However, they can comprise a minor proportion of oily and/or pasty fatty additives, a surfactant and/or a conventional fat-soluble additive/active principle.

Film-Forming Polymers

In the present invention, the term “film-forming polymer” is understood to mean a polymer capable of forming, by itself alone or in the presence of an additional agent which is able to form a film, a macroscopically continuous film which adheres to the eyelashes and preferably a cohesive film and better still a film, the cohesion and the mechanical properties of which are such that the said film can be isolable and handleable in isolation, for example when the said film is produced by casting on a nonstick surface, such as a Teflon- or silicone-treated surface.

Generally, the content of film-forming polymer on a dry basis in the composition can vary from 0.1 to 40% by weight, preferably from 0.5 to 30% by weight and better still from 1 to 10% by weight, with respect to the total weight of the composition. The film-forming polymer can be hydrophilic or lipophilic.

When the film-forming polymer is hydrophilic, it can be provided in the form of a water-soluble polymer or can be provided in dispersion in an aqueous medium.

Mention may be made, among the hydrophilic film-forming polymers which can be used in the composition of the present invention, of synthetic polymers of radical type or of polycondensate type, polymers of natural origin, and their mixtures.

Mention may be made, as examples of water-soluble film-forming polymers, of:
proteins, such as proteins of plant origin, such as wheat or soya protein; proteins of animal origin, such as keratins, for example keratin hydrolysates and sulphonic keratins;
cellulose polymers, such as hydroxyethylcellulose, hydroxypropylcellulose, methylcellulose, ethylhydroxyethylcellulose, carboxymethylcellulose and quaternized cellulose derivatives;
acrylic polymers or copolymers, such as polyacrylates or polymethacrylates;
vinyl polymers, such as polyvinylpyrrolidones, copolymers of methyl vinyl ether and of malic anhydride, the copolymer of vinyl acetate and of crotonic acid, copolymers of vinylpyrrolidone and of vinyl acetate, copolymers of vinylpyrrolidone and of caprolactam, or polyvinyl alcohol;
anionic, cationic, amphoteric or nonionic chitin or chitosan polymers;
gums arabic, guar gum, xanthan derivatives or karaya gum;
alginates and carrageenans;
glycoaminoglycans, hyaluronic acid and its derivatives;
shellac resin, gum sandarac, dammars, elemis or copals;
deoxyribonucleic acid;
mucopolysaccharides, such as chondroitin sulphates;
and their mixtures.

The film-forming polymer can also be present in the composition in the form of particles in dispersion in an aqueous phase, generally known under the name of latex or pseudolatex. The techniques for preparing these dispersions are well known to a person skilled in the art.

Use may be made, as aqueous film-forming polymer dispersion, of acrylic dispersions, sold under the names Neocryl XK-90®, Neocryl A-1070®, Neocryl A-1090®, Neocryl BT-62®, Neocryl A-1079® and Neocryl A-523® by Avecia-Neoresins, Dow Latex 432® by Dow Chemical, Daitosol 5000 AD® or Daitosol 5000 SJ® by Daito Kasey Kogyo; Syntran 5760® by Interpolymer or Allianz Opt® by Röhm & Haas, or also aqueous dispersions of polyurethane, sold under the names Neorez R-981® and Neorez R-974® by Avecia-Neoresins, Avalure UR-405®, Avalure UR-410®, Avalure UR-425®, Avalure UR-450®, Sancure 875®, Avalure UR-445® and Sancure 2060® by Noveon, Impranil 85® by Bayer or Aquamere H-1511® by Hydromer; sulphopolyesters, sold under the trade name Eastman AQ® by Eastman Chemical Products, vinyl dispersions, such as Mexomere PAM®, aqueous dispersions of poly(vinyl acetate), such as “Vinybran®” from Nisshin Chemical or those sold by Union Carbide, aqueous dispersions of terpolymer of vinylpyrrolidone, dimethylaminopropylmethacrylamide and lauryldimethylmethacrylamidopropylammonium chloride, such as Styleze W from ISP, aqueous dispersions of polyurethane/polyacrylic hybrid polymers, such as those sold under the references “Hybridur®” by Air Products or “Duromer®” by National Starch, dispersions of core/shell type: for example those sold by Atofina under the Kynar reference (core: fluorinated—shell: acrylic) or also those described in the document U.S. Pat. No. 5,188,899 (core: silica—shell: silicone), and their mixtures.

According to a specific embodiment, the composition in accordance with the invention comprises, as hydrophilic film-forming polymers, at least the combination of a cationic polymer and of an anionic polymer.

The cationic polymer can be chosen from quaternary cellulose ether derivatives, copolymers of cellulose with a water-soluble quaternary ammonium monomer, cyclopolymers, cationic polysaccharides, silicone cationic polymers, vinylpyrrolidone/dialkylaminoalkyl acrylate or methacrylate copolymers, which may or may not be quaternized, quaternary polymers of vinylpyrrolidone and of vinylimidazole, polyamidoamines and their blends.

Preferably, the cationic polymer is a hydroxy(C₁-C₄)-alkylcellulose comprising quaternary ammonium groups.

The anionic polymer is advantageously chosen from:

A) homo- or copolymers of acrylic acid or methacrylic acid or their salts, copolymers of acrylic acid and of acrylamide and their salts, sodium salts of poly(hydroxycarboxylic acid)s, such as the copolymers of acrylic acid and of acrylamide sold in the form of their sodium salt under the Reten® names by Hercules, poly(sodium methacrylate) sold under the name Darvan No. 7 by Vanderbilt, or sodium salts of poly(hydroxycarboxylic acid)s, sold under the name Hydagen F® by Henkel;
B) copolymers of acrylic acid or methacrylic acid with a monoethylenic monomer, such as ethylene, styrene, vinyl esters or esters of acrylic acid or methacrylic acid, which are optionally grafted to a polyalkylene glycol, such as polyethylene glycol; the copolymers of this type comprising, in their chain, an acrylamide unit which is optionally N-alkylated and/or -hydroxyalkylated, copolymers of acrylic acid and of C₁-C₄ alkyl methacrylate and terpolymers of vinylpyrrolidone, of acrylic acid and of C₁-C₂₀ alkyl methacrylate;
C) copolymers derived from crotonic acid, such as those comprising, in their chain, vinyl acetate or propionate units and optionally other monomers, such as allyl or methallyl esters, vinyl ethers or vinyl esters of a saturated and linear or branched carboxylic acid comprising a long hydrocarbon chain, such as those comprising at least 5 carbon atoms, it being possible for these polymers to be optionally grafted;
D) polymers derived from maleic acid or anhydride, fumaric acid or anhydride, itaconic acid or anhydride with vinyl esters, vinyl ethers, vinyl halides, phenylvinyl derivatives, acrylic acid and its esters; copolymers of maleic anhydride, citraconic anhydride, itaconic anhydride and of an allyl or methallyl ester optionally comprising an acrylamide group, a methacrylamide group, an α-olefin, acrylic or methacrylic esters, acrylic or methacrylic acids, or vinylpyrrolidone in their chain; the anhydride functional groups are monoesterified or monoamidated;
E) polyacrylamides comprising carboxylate groups;
F) deoxyribonucleic acid;
G) copolymers of at least one dicarboxylic acid, of at least one diol and of at least one difunctional aromatic monomer carrying an —SO₃M group with M representing a hydrogen atom, an ammonium ion NH₄⁺ or a metal ion;
and their blends.

The anionic polymers which are more particularly preferred are chosen from noncrosslinked anionic polymers, such as the monoesterified methyl vinyl ether/maleic anhydride copolymers sold under the name Gantrez ES 425 by ISP, the acrylic acid/ethyl acrylate/N-(tert-butyl)acrylamide terpolymers sold under the name Ultrahold Strong by BASF, the copolymers of methacrylic acid and of methyl methacrylate sold under the name Eudragit L by Röhm Pharma, the vinyl acetate/vinyl tert-butylbenzoate/crotonic acid terpolymers and the crotonic acid/vinyl acetate/vinyl neododecanoate terpolymers sold under the name Resin 28-29-30 by National Starch, the copolymers of methacrylic acid and of ethyl acrylate sold under the name Luvimer MAEX or MAE by BASF, the vinylpyrrolidone/acrylic acid/lauryl methacrylate terpolymers sold under the name Acrylidone LM by ISP, the acrylic or methacrylic acid homopolymers sold, for example, under the name Versicol E 5 or the poly(sodium methacrylate) sold under the name Darvan 7 by Vanderbilt, and their blends.

Preferably, the anionic polymer is a poly(sodium methacrylate).

The film-forming polymer which can be used in the composition according to the invention can also be lipophilic. In this case, it is chosen from radical film-forming polymers and polycondensates.

Radical Film-Forming Polymers

The term “radical film-forming polymer” is understood to mean a polymer obtained by polymerization of monomers comprising unsaturation, in particular ethylenic unsaturation, each monomer being capable of homopolymerizing (unlike polycondensates).

The film-forming polymers of radical type can be in particular vinyl polymers or copolymers, in particular acrylic polymers.

The vinyl film-forming polymers can result from the polymerization of monomers comprising ethylenic unsaturation having at least one acid group and/or esters of these acid monomers and/or amides of these acid monomers.

Use may be made, as monomer carrying an acid group, of α,β-ethylenic unsaturated carboxylic acids, such as acrylic acid, methacrylic acid, crotonic acid, maleic acid or itaconic acid. Use is preferably made of (meth)acrylic acid and crotonic acid and more preferably of (meth)acrylic acid.

The esters of acid monomers are advantageously chosen from esters of (meth)acrylic acid (also known as (meth)acrylates), in particular alkyl (meth)acrylates, especially C₁-C₃₀ alkyl (meth)acrylates, preferably C₁-C₂₀ alkyl (meth)acrylates, aryl (meth)acrylates, in particular C₆-C₁₀ aryl (meth)acrylates, or hydroxyalkyl (meth)acrylates, in particular C₂-C₆ hydroxyalkyl (meth)acrylates.

Mention may be made, among alkyl (meth)acrylates, of methyl methacrylate, ethyl methacrylate, butyl methacrylate, isobutyl methacrylate, 2-ethylhexyl methacrylate, lauryl methacrylate or cyclohexyl methacrylate.

Mention may be made, among hydroxyalkyl (meth)acrylates, of hydroxyethyl acrylate, 2-hydroxypropyl acrylate, hydroxyethyl methacrylate or 2-hydroxypropyl methacrylate.

Mention may be made, among aryl (meth)acrylates, of benzyl acrylate and phenyl acrylate.

The particularly preferred esters of (meth)acrylic acid are alkyl (meth)acrylates.

According to the present invention, the alkyl group of the esters can be either fluorinated or perfluorinated, that is to say that a portion or all of the hydrogen atoms of the alkyl group are replaced by fluorine atoms.

Mention may be made, as amides of acid monomers, for example, of (meth)acrylamides and in particular N-alkyl (meth) acrylamides, especially N—(C₂-C₁₂ alkyl)(meth)acrylamides. Mention may be made, among N-alkyl(meth)acrylamides, of N-ethylacrylamide, N-(t-butyl)acrylamide, N-(t-octyl)acrylamide and N-undecyl-acrylamide.

The vinyl film-forming polymers can also result from the homopolymerization or from the copolymerization of monomers chosen from vinyl esters and styrene monomers. In particular, these monomers can be polymerized with acid monomers and/or their esters and/or their amides, such as those mentioned above.

Mention may be made, as examples of vinyl esters, of vinyl acetate, vinyl neodecanoate, vinyl pivalate, vinyl benzoate and vinyl t-butylbenzoate.

Mention may be made, as styrene monomers, of styrene and α-methylstyrene.

Film-Forming Polycondensates

Mention may be made, among film-forming polycondensates, of polyurethanes, polyesters, polyesteramides, polyamides, epoxy ester resins or polyureas.

The polyurethanes can be chosen from anionic, cationic, nonionic or amphoteric polyurethanes, polyurethane-acrylics, polyurethane-polyvinylpyrrolidones, polyester-polyurethanes, polyether-polyurethanes, polyureas, polyurea-polyurethanes, and their blends.

The polyesters can be obtained in a known way by polycondensation of dicarboxylic acids with polyols, in particular diols.

The dicarboxylic acid can be aliphatic, alicyclic or aromatic. Mention may be made, as examples of such acids, of: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid or 2,6-naphthalenedicarboxylic acid. These dicarboxylic acid monomers can be used alone or as a combination of at least two dicarboxylic acid monomers. The choice is preferably made, among these monomers, of phthalic acid, isophthalic acid or terephthalic acid.

The diol can be chosen from aliphatic, alicyclic or aromatic diols. Use is preferably made of a diol chosen from: ethylene glycol, diethylene glycol, triethylene glycol, 1,3-propanediol, cyclohexanedimethanol or 1,4-butanediol. Use may be made, as other polyols, of glycerol, pentaerythritol, sorbitol or trimethylolpropane.

Polyesteramides can be obtained analogously to the polyesters by polycondensation of diacids with diamines or aminoalcohols. Use may be made, as diamines, of ethylenediamine, hexamethylenediamine, metaphenylenediamine or para-phenylenediamine. Use may be made, as aminoalcohol, of monoethanolamine.

According to an alternative embodiment of the composition according to the invention, the lipophilic film-forming polymer can be a polymer dissolved in a liquid fatty phase comprising oils, such as those described below (the film-forming polymer is then described as a fat-soluble polymer). Preferably, the liquid fatty phase comprises a volatile oil, optionally as a mixture with a non-volatile oil, it being possible for the oils to be chosen from the oils mentioned above.

Mention may be made, as examples of fat-soluble polymer, of copolymers of vinyl ester (the vinyl group being directly connected to the oxygen atom of the ester group and the vinyl ester having a saturated, linear or branched, hydrocarbon radical of 1 to 19 carbon atoms bonded to the carbonyl of the ester group) and of at least one other monomer which can be a vinyl ester (other than the vinyl ester already present), an α-olefin (having from 8 to 28 carbon atoms), an alkyl vinyl ether (the alkyl group of which comprises from 2 to 18 carbon atoms) or an allyl or methallyl ester (having a saturated, linear or branched, hydrocarbon radical of 1 to 19 carbon atoms bonded to the carbonyl of the ester group).

These copolymers can be crosslinked using crosslinking agents which can be either of the vinyl type or of the allyl or methallyl type, such as tetraallyloxyethane, divinylbenzene, divinyl octanedioate, divinyl dodecanedioate and divinyl octadecanedioate.

Mention may be made, as examples of these copolymers, of the following copolymers: vinyl acetate/allyl stearate, vinyl acetate/vinyl laurate, vinyl acetate/vinyl stearate, vinyl acetate/octadecyl vinyl ether, vinyl propionate/allyl laurate, vinyl propionate/vinyl laurate, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% of divinylbenzene, vinyl dimethylpropionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/1-octadecene, crosslinked with 0.2% of divinylbenzene, and allyl propionate/allyl stearate, crosslinked with 0.2% of divinylbenzene.

Mention may also be made, as fat-soluble film-forming polymers, of fat-soluble copolymers and in particular those resulting from the copolymerization of vinyl esters having from 9 to 22 carbon atoms and of alkyl acrylates or methacrylates, the alkyl radicals having from 10 to 20 carbon atoms.

Such fat-soluble copolymers can be chosen from copolymers of poly(vinyl stearate), of poly(vinyl stearate) crosslinked using divinylbenzene, diallyl ether or diallyl phthalate, copolymers of poly(stearyl (meth)acrylate), of poly(vinyl laurate), of poly(lauryl (meth)acrylate), it being possible for these poly(meth)acrylates to be crosslinked using ethylene glycol dimethacrylate or tetraethylene glycol dimethacrylate.

The fat-soluble copolymers defined above are known and are described in particular in Application FR-A-2 232 303; they can have a weight-average molecular weight ranging from 2000 to 500,000 and preferably from 4000 to 200,000.

Mention may also be made, as fat-soluble film-forming polymers which can be used in the invention, of polyalkylenes and in particular copolymers of C₂-C₂₀ alkenes, such as polybutene, alkylcelluloses with a saturated or unsaturated and linear or branched C₁ to C₈ alkyl radical, such as ethylcellulose and propylcellulose, copolymers of vinylpyrrolidone (VP) and in particular copolymers of vinylpyrrolidone and of C₂ to C₄₀ alkene and better still C₃ to C₂₀ alkene. Mention may be made, as examples of VP copolymer which can be used in the invention, of the VP/vinyl acetate, VP/ethyl methacrylate, VP/ethyl methacrylate/methacrylic acid, VP/eicosene, VP/hexadecene, VP/triacontene, VP/styrene or VP/acrylic acid/lauryl methacrylate copolymer or butylated polyvinylpyrrolidone (PVP).

Mention may also be made of silicone resins, generally soluble or swellable in silicone oils, which are crosslinked polyorganosiloxane polymers. The nomenclature of silicone resins is known under the name of “MDTQ”, the resin being described according to the various siloxane monomer units which it comprises, each of the letters “MDTQ” characterizing one type of unit. Mention may be made, as examples of commercially available polymethylsilsesquioxane resins, of those which are sold:
by Wacker under the reference Resin MK, such as Belsil PMS MK;
by Shin-Etsu under the reference KR-220L.

Mention may be made, as siloxysilicate resins, of trimethylsiloxysilicate (TMS) resins, such as those sold under the reference SR1000 by General Electric or under the reference TMS 803 by Wacker. Mention may also be made of trimethylsiloxysilicate resins sold in a solvent, such as cyclomethicone, sold under the names “KF-7312J” by Shin-Etsu or “DC 749” or “DC 593” by Dow Corning.

Mention may also be made of copolymers of silicone resins, such as those mentioned above, with polydimethylsiloxanes, such as the pressure-sensitive adhesive copolymers sold by Dow Corning under the reference BIO-PSA and described in the document U.S. Pat. No. 5,162,410, or the silicone copolymers resulting from the reaction of a silicone resin, such as those described above, and of a diorganosiloxane, such as are described in the document WO 2004/073626.

According to one embodiment of the invention, the film-forming polymer is a film-forming linear ethylenic block polymer which preferably comprises at least one first block and at least one second block having different glass transition temperatures (Tg), the said first and second blocks being connected to one another via an intermediate block comprising at least one constituent monomer of the first block and at least one constituent monomer of the second block.

Advantageously, the first and second blocks of the block polymer are incompatible with one another.

Such polymers are described, for example, in the documents EP 1 411 069 and WO 04/028488.

The lipophilic film-forming polymer can also be present in the composition in the form of particles in dispersion in a nonaqueous phase. Mention may be made, as examples of nonaqueous dispersions of film-forming polymer, of acrylic dispersions in isododecane, such as Mexomere PAP® from Chimex, or dispersions of particles of a grafted ethylenic polymer, preferably an acrylic polymer, in a liquid fatty phase, the ethylenic polymer advantageously being dispersed in the absence of an additional stabilizer at the surface of the particles, such as described in particular in the document WO 04/055081.

The composition according to the invention can comprise a plasticizing agent which promotes the formation of a film with the film-forming polymer. Such a plasticizing agent can be chosen from any compound known to a person skilled in the art as being capable of performing the desired role.

According to a first alternative, the composition in accordance with the invention comprises, as continuous phase, an aqueous phase comprising water and/or at least one water-soluble solvent.

The term “composition comprising an aqueous continuous phase” is understood to mean that the composition exhibits a conductivity, measured at 25° C., of greater than 23 μS/cm (microSiemens/cm), the conductivity being measured, for example, using an MPC227 conductivity meter from Mettler Toledo and an Inlab730 conductivity measurement cell. The measurement cell is immersed in the composition, so as to remove the air bubbles liable to be formed between the 2 electrodes of the cell. The conductivity is read as soon as the value of the conductivity meter has stabilized. A mean is determined over at least 3 successive measurements.

According to a second alternative, the composition in accordance with the invention comprises, as continuous phase, an oily phase comprising at least one oil.

The term “composition comprising an oily continuous phase” is understood to mean that the composition exhibits a conductivity, measured at 25° C., of less than 23 μS/cm (microSiemens/cm), the conductivity being measured, for example, using an MPC227 conductivity meter from Mettler Toledo and an Inlab730 conductivity measurement cell. The measurement cell is immersed in the composition, so as to remove the air bubbles liable to be formed between the 2 electrodes of the cell. The conductivity is read as soon as the value of the conductivity meter has stabilized. A mean is determined over at least 3 successive measurements.

Aqueous Phase

The term “aqueous phase” is understood to mean a phase comprising water and/or at least one water-soluble solvent.

The term “water-soluble solvent” denotes, in the present invention, a compound which is liquid at ambient temperature and which is miscible with water (miscibility in water of greater than 50% by weight at 25° C. and atmospheric pressure).

The water-soluble solvents which can be used in the compositions according to the invention can in addition be volatile.

Mention may in particular be made, among the water-soluble solvents which can be used in the compositions in accordance with the invention, of lower monoalcohols having from 1 to 5 carbon atoms, such as ethanol and isopropanol, glycols having from 2 to 8 carbon atoms, such as ethylene glycol, propylene glycol, 1,3-butylene glycol and dipropylene glycol, C₃ and C₄ ketones and C₂-C₄ aldehydes.

The aqueous phase (water and optionally water-miscible solvent) can be present in the composition in a content ranging from 1% to 95% by weight, with respect to the total weight of the composition, preferably ranging from 5% to 80% by weight and preferentially ranging from 10% to 60% by weight.

When the composition according to the invention comprises an aqueous continuous phase, it preferably comprises an aqueous phase in a content ranging from 35 to 95% by weight, preferably ranging from 40 to 90% by weight.

Emulsifying System

When the composition according to the invention comprises an aqueous phase, it can also comprise an emulsifying system.

The composition in accordance with the invention can comprise emulsifying surface-active agents present in particular in a proportion ranging from 0.1 to 30% by weight, with respect to the total weight of the composition, better still from 1 to 15% by weight and better still from 2 to 10% by weight.

According to the invention, use is generally made of an emulsifier appropriately chosen in order to obtain a wax-in-water or oil-in-water emulsion. Use may in particular be made of an emulsifier having, at 25° C., an HLB balance (hydrophilic-lipophilic balance), within the Griffin meaning, of greater than or equal to 8.

The HLB value according to Griffin is defined in J. Soc. Cosm. Chem., 1954 (volume 5), pages 249-256.

These surface-active agents can be chosen from nonionic, anionic, cationic or amphoteric surface-active agents or surface-active emulsifiers. Reference may be made to the document “Encyclopedia of Chemical Technology, Kirk-Othmer”, volume 22, pp. 333-432, 3^{rd} edition, 1979, Wiley, for the definition of the properties and functions (emulsifying) of surfactants, in particular pp. 347-377 of this reference for the anionic, amphoteric and nonionic surfactants.

The surfactants preferably used in the composition according to the invention are chosen from:

a) nonionic surfactants with an HLB of greater than or equal to 8 at 25° C., used alone or as a mixture; mention may in particular be made of:

monosaccharide esters and ethers, such as the mixture of cetylstearyl glucoside and of cetyl and stearyl alcohols, for example Montanov 68 from Seppic;
oxyethylenated and/or oxypropylenated ethers (which can comprise from 1 to 150 oxyethylene and/or oxypropylene groups) of glycerol; oxyethylenated and/or oxypropylenated ethers (which can comprise from 1 to 150 oxyethylene and/or oxypropylene groups) of fatty alcohols (in particular of C₈-C₂₄ and preferably C₁₂-C₁₈ alcohols), such as the oxyethylenated ether of cetearyl alcohol comprising 30 oxyethylene groups (CTFA name “Ceteareth-30”), the oxyethylenated ether of stearyl alcohol comprising 20 oxyethylene groups (CTFA name “Steareth-20”) and the oxyethylenated ether of the mixture of C₁₂-C₁₅ fatty alcohols comprising 7 oxyethylene groups (CTFA name “C12-15 Pareth-7”), sold under the name Neodol 25-7® by Shell Chemicals;
esters of fatty acid (in particular of C₈-C₂₄ and preferably C₁₆-C₂₂ acid) and of polyethylene glycol (which can comprise from 1 to 150 ethylene glycol units), such as PEG-50 stearate and PEG-40 monostearate, sold under the name Myrj 52P® by ICI Uniquema;
esters of fatty acid (in particular of C₈-C₂₄ and preferably C₁₆-C₂₂ acid) and of oxyethylenated and/or oxypropylenated glycerol ethers (which can comprise from 1 to 150 oxyethylene and/or oxypropylene groups), such as PEG-200 glyceryl monostearate, sold under the name Simulsol 220 ™® by Seppic; polyethoxylated glyceryl stearate comprising 30 ethylene oxide groups, such as the product Tagat S® sold by Goldschmidt, polyethoxylated glyceryl oleate comprising 30 ethylene oxide groups, such as the product Tagat 0® sold by Goldschmidt, polyethoxylated glyceryl cocoate comprising 30 ethylene oxide groups, such as the product Varionic LI 13® sold by Sherex, polyethoxylated glyceryl isostearate comprising 30 ethylene oxide groups, such as the product Tagat L® sold by Goldschmidt, and polyethoxylated glyceryl laurate comprising 30 ethylene oxide groups, such as the product Tagat I® from Goldschmidt;
esters of fatty acid (in particular of C₈-C₂₄ and preferably C₁₆-C₂₂ acid) and of oxyethylenated and/or oxypropylenated sorbitol ethers (which can comprise from 1 to 150 oxyethylene and/or oxypropylene groups), such as polysorbate 20, sold under the name Tween20® by Croda, or polysorbate 60, sold under the name Tween60® by Croda;
dimethicone copolyol, such as that sold under the name Q2-5220® by Dow Corning;
dimethicone copolyol benzoate (Finsolv SLB 101® and 201® from Fintex);
copolymers of propylene oxide and of ethylene oxide, also known as EO/PO polycondensates;
and their mixtures.

The EO/PO polycondensates are more particularly copolymers consisting of polyethylene glycol and polypropylene glycol blocks, such as, for example, polyethylene glycol/polypropylene glycol/polyethylene glycol triblock polycondensates. These triblock polycondensates have, for example, the following chemical structure:

H—(O—CH₂—CH₂)ₐ—(O—CH(CH₃)—CH₂)_{b}—(O—CH₂—CH₂)ₐ—OH

in which formula a ranges from 2 to 120 and b ranges from 1 to 100.

The EO/PO polycondensate preferably has a weight-average molecular weight ranging from 1000 to 15, 000 and better still ranging from 2000 to 13, 000. Advantageously, the said EO/PO polycondensate has a cloud point, at 10 g/l in distilled water, of greater than or equal to 20° C., preferably of greater than or equal to 60° C. The cloud point is measured according to Standard ISO 1065. Mention may be made, as EO/PO polycondensate which can be used according to the invention, of the polyethylene glycol/polypropylene glycol/polyethylene glycol triblock polycondensates sold under the Synperonic® names, such as Synperonic PE/L44® and Synperonic PE/F127®, by ICI.

b) nonionic surfactants with an HLB of less than 8 at 25° C., optionally in combination with one or more nonionic surface-active agents with an HLB of greater than 8 at 25° C., such as mentioned above, such as:
esters and ethers of monosaccharides, such as sucrose stearate, sucrose cocoate, sorbitan stearate and their mixtures, such as Arlatone 2121®, sold by ICI;
oxyethylenated and/or oxypropylenated ethers (which can comprise from 1 to 150 oxyethylene and/or oxypropylene groups) fatty alcohols (in particular of C₈-C₂₄ and preferably C₁₂-C₁₈ alcohols), such as the oxyethylenated ether of stearyl alcohol comprising 2 oxyethylene groups (CTFA name “Steareth-2”);
esters of fatty acids (in particular of C₈-C₂₄ and preferably C₁₆-C₂₂ acid) and of polyol, in particular of glycerol or of sorbitol, such as glyceryl stearate, such as the product sold under the name Tegin M® by Goldschmidt, glyceryl laurate, such as the product sold under the name Imwitor 312® by Hüls, polyglyceryl-2 stearate, sorbitan tristearate or glyceryl ricinoleate;
lecithins, such as soya lecithins (for example Emulmetik 100 J from Cargill or Biophilic H from Lucas Meyer);
the cyclomethicone/dimethicone copolyol mixture sold under the name Q2-3225C® by Dow Corning.

c) anionic surfactants, such as:
salts of C₁₆-C₃₀ fatty acids, in particular those deriving from amines, such as triethanolamine stearate and/or 2-amino-2-methylpropane-1,3-diol stearate;
salts of polyoxyethylenated fatty acids, in particular those deriving from amines or the alkali metal salts, and their mixtures;
phosphoric esters and their salts, such as “DEA oleth-10 phosphate” (Crodafos N 10N from Croda) or monocetyl monopotassium phosphate (Amphisol K from Givaudan);
sulphosuccinates, such as “Disodium PEG-5 citrate lauryl sulfosuccinate” and “Disodium ricinoleamido MEA sulfosuccinate”;
alkyl ether sulphates, such as sodium lauryl ether sulphate;
isethionates;
acylglutamates, such as “Disodium hydrogenated tallow glutamate” (Amisoft HS-21 R®, sold by Ajinomoto) and sodium stearoyl glutamate (Amisoft HS-11 PF®, sold by Ajinomoto), and their mixtures;
soya derivatives, such as potassium soyate;
citrates, such as glyceryl stearate citrate (Axol C 62 Pellets from Degussa);
proline derivatives, such as sodium palmitoyl proline (Sepicalm VG from Seppic) or the mixture of sodium palmitoyl sarcosinate, magnesium palmitoyl glutamate, palmitic acid and palmitoyl proline (Sepifeel One from Seppic);
lactylates, such as sodium stearoyl lactylate (Akoline SL from Karlshamns AB);
sarcosinates, such as sodium palmitoyl sarcosinate (Nikkol sarcosinate PN) or the 75/25 mixture of stearoyl sarcosine and myristoyl sarcosine (Crodasin SM from Croda);
sulphonates, such as sodium C₁₄₋₁₇ sec alkyl sulphonate (Hostapur SAS 60 from Clariant);
glycinates, such as sodium cocoyl glycinate (Amilite GCS-12 from Ajinomoto).

The compositions in accordance with the invention can also comprise one or more amphoteric surfactants, such as N-acylamino acids, for example N-alkylaminoacetates and disodium cocoamphodiacetate, and amine oxides, such as stearamine oxide, or also silicone surfactants, such as dimethicone copolyol phosphates, for example that sold under the name Pecosil PS 100® by Phoenix Chemical. The surfactant which can be used can also be a polymeric surfactant, in particular a thermogelling polymer.

Gelling Agents

The composition according to the invention can comprise at least one gelling agent, the said gelling agent being hydrophilic and present in the aqueous phase.

The hydrophilic gelling agents which can be used in the compositions according to the invention can be chosen from:

homo- or copolymers of acrylic acid or methacrylic acid or their salts and their esters and in particular the products sold under the names Versicol F® or Versicol K® by Allied Colloid, Ultrahold 8® by Ciba-Geigy, polyacrylic acids of Synthalen K type;

copolymers of acrylic acid and of acrylamide, sold in the form of their sodium salt under the Reten® names by Hercules, sodium salts of polyhydroxycarboxylic acids, sold under the name Hydagen F® by Henkel;

copolymers of polyacrylic acids and of alkyl acrylates of Pemulen type;

AMPS (polyacrylamidomethylpropanesulphonic acid partially neutralized with aqueous ammonia and highly crosslinked), sold by Clariant;

AMPS/acrylamide copolymers of Sepigel® or Simulgel® type, sold by Seppic;

copolymers of AMPS and of alkyl methacrylates which are polyoxyethylenated (crosslinked or noncrosslinked);

associative polyurethanes, such as the polymer C₁₆-OE₁₂₀-C₁₆ from Servo Delden (sold under the name SER AD FX1100, molecule comprising a urethane functional group and with a weight-average molecular weight of 1300), OE being an oxyethylene unit, Rheolate 205 comprising a urea functional group, sold by Rheox, or also Rheolate 208 or 204 (these polymers being sold in the pure form), or DW 1206B from Röhm & Haas comprising a C₂₀ alkyl chain and comprising a urethane bond, sold at 20% of active material in water. Use may also be made of solutions or dispersions of these associative polyurethanes, in particular in water or in an aqueous/alcoholic medium. Mention may be made, by way of example of such polymers, of SER AD fx1010, SER AD FX1035 and SER AD 1070 from Servo Delden and Rheolate 255, Rheolate 278 and Rheolate 244, sold by Rheox. Use may also be made of the products DW 1206F and DW 1206J, and also Acrysol RM 184 or Acrysol 44, from Röhm & Haas, or alternatively Borchigel LW 44 from Borchers;

and their mixtures.

Some water-soluble film-forming polymers mentioned above can also act as water-soluble gelling agent.

The hydrophilic gelling agents can be present in the composition according to the invention in a content ranging from 0.05 to 40% by weight, with respect to the total weight of the composition, preferably from 0.5 to 20% by weight and better still from 1 to 15% by weight.

Oils

The composition in accordance with the invention can comprise one or more oils.

The term “oil” is understood to mean a nonaqueous body which is liquid at ambient temperature and atmospheric pressure. The oil can be volatile or nonvolatile.

The term “volatile oil” is understood to mean, within the meaning of the invention, any nonaqueous medium capable of evaporating on contact with the eyelashes in less than one hour at ambient temperature and atmospheric pressure. The volatile oil or oils of the invention are volatile cosmetic oils which are liquid at ambient temperature and which have a non-zero vapour pressure, at ambient temperature and atmospheric pressure, ranging from 0.13 Pa to 40, 000 Pa (10⁻³ to 300 mmHg), in particular ranging from 1.3 Pa to 13, 000 Pa (0.01 to 100 mmHg) and more particularly ranging from 1.3 Pa to 1300 Pa (0.01 to 10 mmHg). The term “nonvolatile oil” is understood to mean an oil which remains on the eyelashes at ambient temperature and atmospheric pressure for at least several hours and which has in particular a vapour pressure of less than 10⁻³ mmHg (0.13 Pa).

The oil can be present in the composition in a content ranging from 1 to 85% by weight, preferably 5 to 80% by weight, with respect to the total weight of the composition. When the composition according to the invention comprises an oily continuous phase, it preferably comprises an oily phase in a content ranging from 35 to 90% by weight, preferably ranging from 40 to 80% by weight. The composition can comprise volatile oils and/or nonvolatile oils, and their mixtures.

The volatile oils can be hydrocarbon oils, silicone oils, fluorinated oils or their mixtures.

The term “hydrocarbon oil” is understood to mean an oil comprising mainly hydrogen and carbon atoms and optionally oxygen, nitrogen, sulphur or phosphorus atoms. Volatile hydrocarbon oils can be chosen from hydrocarbon oils having from 8 to 16 carbon atoms, in particular branched C₈-C₁₆ alkanes, such as C₈-C₁₆ isoalkanes of petroleum origin (also known as isoparaffins), such as isododecane (also known as 2,2,4,4,6-pentamethylheptane), isodecane or isohexadecane, for example the oils sold under the “Isopars®” or “Permethyls®” trade names, branched C₈-C₁₆ esters, isohexyl neopentanoate, and their mixtures. Other volatile hydrocarbon oils, such as petroleum distillates, in particular those sold under the “Shell Solt®” name by Shell, can also be used.

Use may also be made, as volatile oils, of volatile silicones, such as, for example, volatile linear or cyclic silicone oils, in particular those having a viscosity ≦6 centistokes (6×10⁻⁶ m²/s) and having in particular from 3 to 6 silicon atoms, these silicones optionally comprising one or more alkyl or alkoxy groups having 1 or 2 carbon atoms. Mention may in particular be made, as volatile silicone oil which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

The composition in accordance with the invention can also comprise at least one nonvolatile oil which can be chosen in particular from nonvolatile hydrocarbon oils and/or silicone oils and/or fluorinated oils.

Mention may in particular be made, as nonvolatile hydrocarbon oil, of:

hydrocarbon oils of vegetable origin, such as triglycerides composed of esters of fatty acids and of glycerol, the fatty acids of which can have varied chain lengths from C₄ to C₂₄, it being possible for these chains to be linear or branched and saturated or unsaturated; these oils are in particular wheat germ, sunflower, grape seed, sesame, maize, apricot kernel, castor, shea, avocado, olive, soya bean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, candlenut, passionflower or musk rose oil; or triglycerides of caprylic/capric acids, such as those sold by Stéarineries Dubois or those sold under the names Miglyol 810®, 812® and 818® by Dynamit Nobel;

synthetic ethers having from 10 to 40 carbon atoms;

linear or branched hydrocarbons of mineral or synthetic origin, such as liquid petrolatum, polydecenes, hydrogenated polyisobutene, such as Parleam oil, squalane, and their mixtures;

synthetic esters, such as the oils of formula R₁COOR₂ in which R₁ represents the residue of a linear or branched fatty acid comprising from 1 to 40 carbon atoms and R₂ represents a hydrocarbon chain, in particular a branched hydrocarbon chain, comprising from 1 to 40 carbon atoms, provided that R₁+R₂ is ≧10, such as, for example, Purcellin oil (cetearyl octanoate), isopropyl myristate, isopropyl palmitate, C₁₂ to C₁₅ alkyl benzoate, hexyl laurate, diisopropyl adipate, isononyl isononanoate, 2-ethylhexyl palmitate, isostearyl isostearate, octanoates, decanoates or ricinoleates of alcohols or of polyalcohols, such as propylene glycol dioctanoate; hydroxylated esters, such as isostearyl lactate or diisostearyl malate; and pentaerythritol esters;

fatty alcohols which are liquid at ambient temperature comprising a branched and/or unsaturated carbon chain having from 12 to 26 carbon atoms, such as octyldodecanol, isostearyl alcohol, oleyl alcohol, 2-hexyldecanol, 2-butyloctanol or 2-undecylpentadecanol;

higher fatty acids, such as oleic acid, linoleic acid or linolenic acid;

and their mixtures.

The nonvolatile silicone oils which can be used in the compositions in accordance with the invention can be polydimethylsiloxanes (PDMSs) which are nonvolatile, polydimethylsiloxanes comprising pendent alkyl or alkoxy groups and/or alkyl or alkoxy groups at the end of the silicone chain, groups each having from 2 to 24 carbon atoms, phenylated silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

The fluorinated oils which can be used in the compositions in accordance with the invention are in particular fluorosilicone oils, fluorinated polyethers or fluorinated silicones, such as described in the document EP-A-847 752.

The content of nonvolatile oil in the compositions in accordance with the invention can vary from 0.01 to 30% by weight, in particular from 0.1 to 25% by weight and better still from 0.1 to 20% by weight, with respect to the total weight of the composition.

Colouring Material

The compositions in accordance with the invention can also comprise at least one colouring material, such as pulverulent materials, fat-soluble dyes or water-soluble dyes.

The pulverulent colouring materials can be chosen from pigments and pearlescent agents.

The pigments can be white or coloured, inorganic and/or organic and coated or uncoated. Mention may be made, among inorganic pigments, of titanium dioxide, optionally treated at the surface, zirconium, zinc or cerium oxides, and also iron or chromium oxides, manganese violet, ultramarine blue, chromium hydrate and ferric blue. Mention may be made, among organic pigments, of carbon black, pigments of D & C type, and lakes, based on cochineal carmine, of barium, strontium, calcium or aluminium.

The pearlescent agents can be chosen from white pearlescent pigments, such as mica covered with titanium oxide or with bismuth oxychloride, coloured pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

The fat-soluble dyes are, for example, Sudan red, D&C Red 17, D&C Green 6, β-carotene, soya bean oil, Sudan brown, D&C Yellow 11, D&C Violet 2, D&C Orange 5, quinoline yellow or annatto.

These colouring materials can be present in a content ranging from 0.01 to 30% by weight, with respect to the total weight of the composition.

Fillers

The compositions in accordance with the invention can additionally comprise at least one filler.

The fillers can be chosen from those well known to a person skilled in the art and commonly used in cosmetic compositions. The fillers can be inorganic or organic and lamellar or spherical. Mention may be made of talc, mica, silica, kaolin, powders formed of polyamide, such as Nylon®, sold under the name Orgasol® by Atochem, of poly-β-alanine and of polyethylene, powders formed of tetrafluoroethylene polymers, such as Teflon®, lauroyllysine, starch, boron nitride, hollow polymer microspheres which are expanded, such as those of poly(vinylidene chloride)/acrylonitrile, for example those sold under the name Expancel® by Nobel Industrie, acrylic powders, such as those sold under the name Polytrap® by Dow Corning, particles formed of polymethyl methacrylate and silicone resin microbeads (Tospearls® from Toshiba, for example), precipitated calcium carbonate, magnesium carbonate, basic magnesium carbonate, hydroxyapatite, hollow silica microspheres (Silica Beads® from Maprecos), glass or ceramic microcapsules, or metal soaps derived from organic carboxylic acids having from 8 to 22 carbon atoms and in particular from 12 to 18 carbon atoms, for example zinc stearate, magnesium stearate, lithium stearate, zinc laurate or magnesium myristate.

Use may also be made of a compound capable of swelling when heated and in particular of heat-expandable particles, such as nonexpanded microspheres formed of vinylidene chloride/acrylonitrile/methyl methacrylate copolymer or of copolymer of homopolymer of acrylonitrile, such as, for example, those sold respectively under the references Expancel® 820 DU 40 and Expancel® 007WU by Akzo Nobel.

The fillers can represent from 0.1 to 25% by weight, in particular from 1 to 20% by weight, with respect to the total weight of each composition.

Fibres

The compositions in accordance with the invention can additionally comprise fibres which make possible an improvement in the lengthening effect.

The term “fibre” should be understood as meaning an object with a length L and a diameter D such that L is much greater than D, D being the diameter of the circle in which the cross section of the fibre is framed. In particular, the L/D ratio (or aspect ratio) is chosen within the range from 3.5 to 2500, in particular from 5 to 500 and more particularly from 5 to 150.

The fibres which can be used in the composition of the invention can be fibres of synthetic or natural and inorganic or organic origin. They can be short or long, individual or organized, for example plaited, and hollow or solid. They can have any shape and can in particular be circular or polygonal (square, hexagonal or octagonal) in cross section, according to the specific application envisaged. In particular, their ends are blunted and/or polished to prevent injury.

In particular, the fibres have a length ranging from 1 μm to 10 mm, in particular from 0.1 mm to 5 mm and more particularly from 0.3 mm to 3.5 mm. Their cross section can be included within a circle with a diameter ranging from 2 nm to 500 μm, in particular ranging from 100 nm to 100 μm and more particularly ranging from 1 μm to 50 μm. The weight or count of the fibres is often given in denier or decitex and represents the weight in grams per 9 km of yarn. The fibres according to the invention can in particular have a count chosen within the range from 0.15 to 30 denier and in particular from 0.18 to 18 denier.

The fibres which can be used in the composition of the invention can be chosen from rigid or nonrigid fibres. They can be of synthetic or natural and inorganic or organic origin.

Furthermore, the fibres may or may not be surface treated, may or may not be coated and may or may not be coloured.

Mention may be made, as fibres which can be used in the composition according to the invention, of fibres which are not rigid, such as polyamide (Nylon®) fibres, or fibres which are rigid, such as polyimideamide fibres, for example those sold under the Kermel® or Kermel Tech® names by Rhodia, or poly(p-phenylene terephthalamide) (or aramid) fibres, sold in particular under the Kevlar® name by DuPont de Nemours.

The fibres can be present in the composition according to the invention in a content ranging from 0.01% to 10% by weight, with respect to the total weight of the composition, in particular from 0.1% to 5% by weight and more particularly from 0.3% to 3% by weight.

Cosmetic Active Principles

Mention may in particular be made, as cosmetic active principles which can be used in the compositions in accordance with the invention, of antioxidants, preservatives, fragrances, neutralizing agents, emollients, moisturizing agents, vitamins and screening agents, in particular sunscreens.

Of course, a person skilled in the art will take care to choose the optional additional additives and/or their amounts so that the advantageous properties of the composition according to the invention are not, or not substantially, detrimentally affected by the envisaged addition.

Combination

The combination according to the present invention comprises at least one applicator for the cosmetic composition in accordance with the invention. The applicator comprises means which make it possible to smooth and/or separate the eyelashes or eyebrows, in particular in the form of teeth, bristles or other raised patterns.

The composition is applied using the applicator, also known as application means, described more particularly below.

The combination according to the invention is preferably provided in the form of one and the same packaging. It can thus be provided as a container delimiting at least one compartment or reservoir which comprises the composition according to the invention, the said compartment being optionally closed by a closure part, and at least one application means for the composition.

The closure part can be coupled to the container by screwing. Alternatively, the closure part and the container are coupled other than by screwing, in particular via a bayonet mechanism, by latching or by clamping. The term “latching” is understood to mean in particular any system which involves surmounting a flange or ring of material by elastic deformation of a portion, in particular of the closure part, and then by returning to the elastically unstressed position of the said portion after the flange or ring has been surmounted.

The container can be at least partly made of thermoplastic. Mention may be made, as examples of thermoplastics, of polypropylene or polyethylene.

Alternatively, the container is made of nonthermoplastic material, in particular of glass or of metal (or alloy).

The container is preferably equipped with a wringer positioned in the vicinity of at least one opening of the container. Such a wringer makes it possible to wipe the applicator and optionally the rod to which it may be integrally attached. Such a wringer is described, for example, in Patent FR 2 792 618. The wringer can, for example, comprise a block of a cellular material, such as an open-cell or closed-cell foam, with or without flocking. In an alternative form, the wringer can comprise a non-cellular material which is optionally flocked, for example an elastomer or a polyolefin. In this case in particular, the wringer can, for example, comprise at least one slit and/or comprise a lip arranged in order to wring the rod.

According to a particularly preferred embodiment, the make-up combination comprises a reservoir comprising the composition according to the invention, the reservoir being provided with the application means (or applicator) for the composition described below.

Application Means

The application means, or applicator, can be used to apply the composition to a region to be made up. The applicator can also be used for the finishing of the make-up, on a made-up region or a region charged with product using another applicator.

The composition according to the invention can be withdrawn from a container by immersing the application element in the latter.

The applicatory part can optionally comprise a closure part for a container comprising the composition according to the invention.

The application element is arranged in order to apply a product to the eyelashes and can comprise, for example, a brush or a comb.

The application element can also be arranged in order to apply a product in the form of an eyeliner and can comprise, for example, a fine brush.

The brush can comprise a twisted core and bristles taken between the turns of the core, or be made in another way again.

The comb is, for example, made from a single component by moulding of plastic.

The application element can be magnetic.

The applicator may or may not comprise a reservoir comprising the composition.

When the applicator does not comprise a reservoir comprising the composition, the composition is, for example, present in a container and the application element is, for example, charged with composition by being introduced at least partially into this container. The container may or may not comprise a wringing device.

The reservoir may or may not be attached in removable fashion to the applicator. When the reservoir is permanently attached to the applicator, in order to feed the latter with product, the wall of the reservoir serves, for example, for the grasping of the applicator.

The combination according to the invention can also comprise at least two different application elements which can be selectively mounted on the applicator. The combination comprises, for example, several different applicatory parts or several application elements arranged so as to be mounted on a part of the applicator.

A better understanding of the combination can be achieved on reading the detailed description which will follow of nonlimiting implementational examples of the latter and on examining the appended drawing, in which:

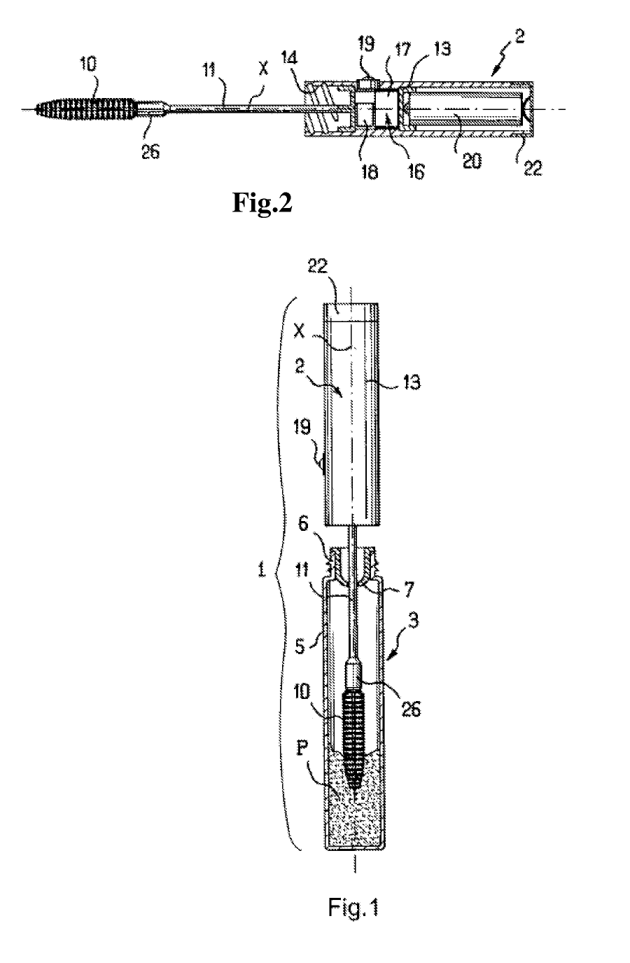

FIG. 1 diagrammatically represents, with partial longitudinal section, a packaging and application system in accordance with an implementational example of the invention.

FIG. 1, to which reference is now made, illustrates a packaging and application system 1 in accordance with an embodiment described in Application WO2006/090343. According to this embodiment, the packaging and application combination comprises an applicator 2 and a container 3 comprising a supply of a make-up product P, in the case in point a composition according to the invention, for example mascara.

The container 3 is conventional in the example illustrated and comprises a body 5 provided with a neck 6 provided externally with a thread. A wringing device 7 is attached inside the neck 6.

The applicator 2 comprises an application element 10 composed, in the example under consideration, of a mascara brush. The application element 10 is mounted at the end of a rod 11, the other end of which is integrally attached to a housing 13 comprising an end part 14 arranged in order to be screwed onto the neck 6 and to thus close the container 3 in a leaktight manner.

The brush 10 comprises, for example, bristles, the ends of which are positioned as helical layers.

The rod can be produced with a uniform or a nonuniform transverse cross section.

When the application device is configured in the form of a comb, the latter can be in accordance with what is described in particular in the publications US 2003-0089379-A1, U.S. Pat. No. 6,655,390, U.S. Pat. No. 6,814,084, U.S. Pat. No. 6,675,814, U.S. Pat. No. 6,581,610, U.S. Pat. No. 6,546,937, U.S. Pat. No. 6,539,950, U.S. Pat. No. 6,412,496 or U.S. Pat. No. 6,343,607, this list not being limiting.

The invention is not limited to a specific application element and the latter can in particular be provided with a means which makes it possible to heat the product and/or the eyelashes during application. This can make it possible to modify the rheology of the composition during application.

Various modifications can be made to the implementational examples which have just been described without departing from the scope of the present invention.

Likewise, the application element can in particular be provided with a means which makes it possible to vibrate the product and/or the eyelashes during the application.

When the container comprises a wringing device through which the application element is withdrawn, the application element can also be subjected to vibrations when passing through the wringing device, which can make it possible to obtain a wringing of the application element different from that which exists in the absence of vibration of the application element. The user can thus, for example, choose between at least two degrees of wringing of the application element, depending on whether or not the application element vibrates when passing through the wringing device.

In addition, it proves to be more advantageous to vibrate the applicator than the wringing device, since the vibrations of the applicator can be of use for the application also.

The method for making up or caring for the eyelashes according to the invention can comprise the adjusting by the user of a vibration frequency and/or the adjusting of a vibration amplitude, for example by acting on an adjusting device. The vibration amplitude for the application element during application is, for example, less than or equal to 5 mm, better still less than or equal to 3 mm, microvibrations of the application element being preferable to vibrations with a higher amplitude.

The vibrations can be obtained in various ways, mechanically, hydraulically, pneumatically, electronically or electromechanically. The vibrating source can comprise, for example, a motor driving a counterweight, an eccentric, an electromagnet or a piezoelectric or mechanical vibrator.

The vibrating source and the applicatory part can be in localized or extended contact, according to, for example, the amplitude, the frequency and the orientation of the vibrations. The frequency of the vibrations is, for example, greater than or equal to 20 Hz.

The vibrations can be orientated transversely to the longitudinal axis of the application element or parallel to the latter or in another way again.

According to a specific embodiment, the vibrating unit is mounted in removable fashion relative to the remainder of the application unit. This makes it possible to use a vibrating unit in combination with different applicatory parts, in order, for example, to treat in a different way according to the applicatory parts selected.

The vibrating source can be present permanently on the applicator or, in an alternative form, form part of a vibrating unit which can be attached in removable fashion to an applicatory part of the applicator.

The combination can thus comprise a vibrating unit and several applicatory parts in association with different application elements or products. The combination comprises, for example, a vibrating unit and at least two applicatory parts chosen from applicatory parts intended for making up or caring for the eyelashes.

A better understanding of the combination can be obtained on reading the detailed description which will follow of nonlimiting implementational examples of the latter and on examining the appended drawing, in which:

FIG. 2 represents in isolation the applicator of FIG. 1, with partial and schematic longitudinal section.

FIG. 2, to which reference is made, illustrates a packaging and application system. The applicator 2 is according to FIG. 1.

The housing 13 houses a vibrating source which makes it possible to vibrate the application element 10 during the application of the make-up product and/or during withdrawal of product from the container and/or extraction of the application element.

The vibrating source comprises, in the example under consideration, a vibrator 16 composed of a motor 17 and of a counterweight 18 driven in rotation by the motor and having a centre of gravity eccentric with respect to the axis of rotation. The motor 17 is fed electrically by an energy source 20, such as, for example, a pencil battery, housed in the housing 13 in the example under consideration and connected electrically to the motor via a switch 19.

The housing 13 comprises a removable lid 22 which makes possible the insertion and the replacement of the battery 20 and which can provide electrical contact with the latter.

In the example under consideration, the axis of rotation of the motor 17 coincides substantially with the longitudinal axis X of the applicator, so that the vibrations are produced substantially perpendicularly to the axis X. These vibrations are propagated along the rod 11 and cause the application element 10 to vibrate substantially perpendicularly to the axis X during application of the product to the eyelashes.

In this figure, the brush has been represented highly diagrammatically in order to clearly illustrate the fact that the invention is not limited to a specific application element.

The brush 10 comprises, for example, bristles, the ends of which are positioned as helical layers. The oscillation of the brush 10 makes it possible to obtain a relative movement of the bristles of the brush 10 along the eyelashes and thus to smooth the product at the surface of the latter and/or to orientate optional fibres present in the product P. The vibrations of the brush 10 can also facilitate the separation of the eyelashes.

The motor 17 can be started during the application of the product to the eyelashes, whether during the initial application of the product or after the initial application, in order to complete the make-up. The user can also vibrate the brush when it is immersed in the container 3, so as, for example, to facilitate the charging of the brush with product, for example to obtain a more homogeneous charge. The user can also vibrate the brush 10 when it surmounts the wringing device 7.

The rod can be produced with a uniform or nonuniform transverse cross section. Preferably, the rod 11 is flexible, which can increase the amplitude of the vibrations of the brush 10, it being possible for a person skilled in the art to choose the dimensions of the rod according, for example, to the nature of the application element, of the product and of the treatment to be carried out.

Various modifications may be made to the implementational examples which have just been described without departing from the scope of the present invention. For example, the vibrating source can comprise a vibrator other than an electric motor driving in rotation a counterweight and other than a piezoelectric vibrator. The vibrating source can in particular comprise any electromechanical, pneumatic, hydraulic, mechanical or electronic system capable of producing vibrations.

The vibrating source can comprise means for controlling the vibrations other than a simple on-off switch and in particular can comprise mechanical or electronic control means which make it possible to adjust the amplitude and/or the frequency of the vibrations. The control means can, for example, comprise a rotary or linear commutator or potentiometer which makes it possible to select at least two rotational speeds of the electric motor in the case where the vibrator comprises such a motor.

The vibrating source can comprise more than one vibrator and, for example, two vibrators arranged in order to produce oscillations in different directions. In this case, the vibrating source can also comprise, for example, a selector which makes it possible to select the vibrator or vibrators which it is desired to bring into operation.

The vibrating source can, if desired, be orientated by the user so as to vibrate the application element with the vibrations of the desired orientation.

The vibrating source can comprise an energy source which can be other than a battery and in particular can comprise one or more accumulators or capacitors. If appropriate, the vibrating source can be arranged so as to be able to be recharged with electricity by being rested on a socket outlet.

If appropriate, the vibrating source can be fed by the mains supply via an optional transformer.

The vibrating source can be mounted in multiple ways in a corresponding receptacle of the applicator and the mounting of the vibrating source is, for example, designed so as to promote the transfer of vibrations towards the application element.

The vibrating source can be brought into operation in other ways still than those which have just been described.

The application elements can be of any type, in particular comprising capillary slits, or others still.

The application elements can be produced in various ways, in particular by moulding, injection overmoulding, fastening or twisting.

The application elements can be single-use application elements, if appropriate.

The application elements can be attached by any means to the applicatory part, in particular by adhesive bonding, welding, punching, latching, screwing, with magnets, by friction, by coupling of Velcro® type or by clamping between jaws or arms of a clamp.

The application element can be driven in rotation, if appropriate, for example as described in U.S. Pat. No. 4,937,326, U.S. Pat. No. 4,922,934 and U.S. Pat. No. 6,565,276, the contents of which are incorporated in the present document by reference.

Finally, a subject-matter of the present invention is a combination for making up or caring for the eyelashes or eyebrows, comprising:

at least one composition comprising, in a physiologically acceptable medium, at least one active principle capable of inducing and/or stimulating the growth of the eyelash or eyebrow, and

at least one applicator for the composition, the said applicator comprising means which make it possible to smooth and/or separate the eyelashes or eyebrows, in particular in the form of teeth, bristles or other raised patterns, and vibration means.

Preferably, the active principle is chosen from arginines, their organic or inorganic acid salts and their derivatives, the compounds of formula (I) and the compounds of formula (II).

Preferably, the composition comprises at least one compound chosen from arginines, their organic or inorganic acid salts and their derivatives, at least one compound of formula (I) and at least one compound of formula (II). More preferably, the composition comprises at least one compound chosen from arginines, their organic or inorganic acid salts and their derivatives, at least one compound of formula (I), at least one compound of formula (II) and citric acid.

The following examples serve to illustrate the compositions according to the invention without, however, exhibiting a limiting nature. The amounts indicated in the various formulations are expressed as percentages by weight, with respect to the total weight of the composition.

### EXAMPLES

### Example 1

### Emulsion Mascara

| | Amount |
|---|---|
| Ingredient | (% by weight) |
| EDTA | 0.2 |
| L-Arginine | 0.05 |
| L-Serine | 0.05 |
| L-Glutamic acid | 0.05 |
| Ceramide | 0.05 |
| Citric acid | 0.05 |
| Madecassoside | 0.3 |
| Carnauba wax | 3 |
| Beeswax | 3.8 |
| Cetyl alcohol | 2 |
| Paraffin wax | 12 |
| Hydrogenated jojoba oil | 0.25 |
| Palm oil | 0.25 |
| Black iron oxide | 7.14 |
| Preservatives | q.s. |
| Ethylenediamine/stearyl dimer dilinoleate | 0.5 |
| copolymer (Uniclear 100 VG from Arizona | |
| Chemical) | |
| Hydroxyethylcellulose | 0.75 |
| Gum arabic | 0.63 |
| Sodium hyaluronate | 0.1 |
| Cellulose fibres | 0.5 |
| Crosslinked ethyl acrylate/methyl | 10 |
| methacrylate (80/20) copolymer, as a 50% | |
| aqueous dispersion (Daitosol 5000 AD from | |
| Daito Kasei Kogyo) | |
| Water | 46.46 |
| Steareth-2 (Brij 72 from Croda) | 2.1 |
| Potassium cetyl phosphate (Amphisol K | 7 |
| from DSM Nutritional Products) | |
| Polysorbate-21 (Tween 21 from Croda) | 0.5 |
| Panthenol | 0.5 |

A panel of 32 women was chosen for evaluating this mascara.

They applied this mascara for 4 weeks: with a vibrating brush over one eye and without the vibration over the other eye (according to a randomization plan).

A cosmetic clinical evaluation was carried out by an ophthalmologist at T0 and after using the mascara for 4 weeks.

After using for 4 weeks, as is shown in the table below, the ophthalmologist demonstrates a significant change in favour of the mascara, applied with and without vibrations, with regard to all the criteria evaluated by the ophthalmologist, namely:

the index for general state of the eyelashes: with the evenness of the surface of the eyelashes, the sheen of the surface of the eyelashes, the macroscopic sheen and the suppleness of the eyelashes;

the eyelash density;

the length of the eyelashes; and

the thickness of the eyelashes.

Furthermore, a significant advantage in favour of the mascara applied with vibrations emerges for the following criteria:

the index for general state of the eyelashes: with the evenness of the surface of the eyelashes, the sheen of the surface of the eyelashes, the macroscopic sheen and the suppleness of the eyelashes;

the eyelash density; and

the length of the eyelashes.

Results of the Clinical Evaluation by the Ophthalmologist:

| | | |
|---|---|---|
| N = 32 | Without vibration | With vibrations |

| Clinical evaluation | | T = | | T = |
|---|---|---|---|---|
| of the eyelashes | T0 | 4 weeks | T0 | 4 weeks |
| Surface evenness (1) | 1.28 | 2.75* | 1.25 | 2.94* |
| (score from 0 to 5) | | | | |
| Surface sheen (2) | 1.72 | 2.69* | 1.72 | 2.88* |
| (score from 0 to 5) | | | | |
| Macroscopic sheen | 1.97 | 2.81* | 2.00 | 2.88* |
| (3) (score from 0 to 5) | | | | |
| Suppleness (4) | 2.84 | 5.06* | 2.88 | 5.91* |
| (score from 0 to 10) | | | | |
| General index | 7.81 | 13.31* | 7.84 | 14.59* |
| (i.e. 1 + 2 + 3 + 4) | | | | |
| Eyelash density | 1.53 | 2.25* | 1.53 | 2.94* |
| (score from 0 to 5) | | | | |
| Length | 2.09 | 2.75* | 2.09 | 2.91* |
| (score from 0 to 5) | | | | |
| Thickness | 1.50 | 2.13* | 1.50 | 2.19* |
| (score from 0 to 5) | | | | |

*significant improvement

### Example 2

### Emulsion Mascara

| | Amount |
|---|---|
| Ingredient | (% by weight) |
| EDTA | 0.2 |
| L-Arginine | 0.05 |
| L-Serine | 0.05 |
| L-Glutamic acid | 0.05 |
| Ceramide | 0.05 |
| Citric acid | 0.05 |
| Madecassoside | 0.5 |
| Carnauba wax | 3 |
| Beeswax | 3.8 |
| Cetyl alcohol | 2 |
| Paraffin wax | 12 |
| Hydrogenated jojoba oil | 0.25 |
| Palm oil | 0.25 |
| Titanium oxide | 0.5 |
| Preservatives | q.s. |
| Ethylenediamine/stearyl dimer dilinoleate | 0.5 |
| copolymer (Uniclear 100 VG from Arizona | |
| Chemical) | |
| Hydroxyethylcellulose | 0.75 |
| Gum arabic | 0.63 |
| Sodium hyaluronate | 0.1 |
| Cellulose fibres | 0.5 |
| Crosslinked ethyl acrylate/methyl | 10 |
| methacrylate (80/20) copolymer, as a 50% | |
| aqueous dispersion (Daitosol 5000 AD from | |
| Daito Kasei Kogyo) | |
| Water | 52.9 |
| Steareth-2 (Brij 72 from Croda) | 2.1 |
| Potassium cetyl phosphate (Amphisol K | 7 |
| from DSM Nutritional Products) | |
| Polysorbate-21 (Tween 21 from Croda) | 0.5 |
| Panthenol | 0.5 |

### Example 3

### Care Product for the Eyelash

| | Amount |
|---|---|
| Ingredient | (% by weight) |
| L-Arginine | 0.05 |
| Citric acid | 0.05 |
| Madecassoside | 0.3 |
| Preservatives | q.s. |
| Hydroxyethylcellulose | 2 |
| Polysorbate-21 (Tween 21 from Croda) | 0.5 |
| Sodium hyaluronate | 0.1 |
| Ethanol | 8 |
| Water | q.s. for 100 |

A panel of 32 women was chosen for evaluating this care product for the eyelash. The results are presented in the table below.

They applied this care product for the eyelash under their normal mascara in the morning and this care product for the eyelash, alone, a further time in the evening, for 4 weeks (T5 to T8 weeks in the table), after observation of the change in their eyelashes after using their normal mascara for 4 weeks (T0 and T4 weeks in the table).

A cosmetic clinical evaluation was carried out by an ophthalmologist at T0 and after using the care product for the eyelash for 4, 5, 6, 7 and 8 weeks.

Results of the Clinical Evaluation by the Ophthalmologist of the Care Product for the Eyelash:

| | | | Normal mascara + care product for | | | |
|---|---|---|---|---|---|---|
| | | | the eyelash (care product applied | | | |
| | Normal | | under the mascara in the morning | | | |
| | mascara only | | and alone in the evening) | | | |
| Clinical evaluation | | T = 4 | T = 5 | T = 6 | T = 7 | T = 8 |
| of the eyelashes | T0 | weeks | weeks | weeks | weeks | weeks |
| Surface evenness (1) | 1.19 | 1.13 | 2.09* | 2.97* | 3.59* | 4.13* |
| (score from 0 to 5) | | | | | | |
| Surface sheen (2) | 1.22 | 1.28 | 2.22* | 3.19* | 3.63* | 4.31* |
| (score from 0 to 5) | | | | | | |
| Macroscopic sheen (3) | 1.84 | 1.72 | 2.5* | 3.34* | 3.75* | 4.22* |
| (score from 0 to 5) | | | | | | |
| Suppleness (4) | 3.34 | 2.91 | 5.03* | 6.63* | 7.63* | 8.25* |
| (score from 0 to 10) | | (significant | | | | |
| | | improvement T = | | | | |
| | | 4 weeks vs T0) | | | | |
| General index | 7.59 | 7.03 | 11.84* | 16.13* | 18.59* | 20.91* |
| (i.e., 1 + 2 + 3 + 4) | | | | | | |

*significant improvement

It emerges from the above table that the use of the care product for the eyelash improves from week to week and significantly all the clinical criteria evaluated by the ophthalmologist to achieve a 3 times greater improvement in the general state of the eyelashes after using for 1 month (T=4 weeks vs T=8 weeks).

### Example 4

### Serum for the Eyelashes

| | Amount |
|---|---|
| Ingredient | (% by weight) |
| Sodium hydroxide | 0.02 |
| L-Arginine | 0.05 |
| L-Serine | 0.05 |
| L-Glutamic acid | 0.05 |
| Ceramide | 0.01 |
| Citric acid | 0.05 |
| Madecassoside | 0.3 |
| 2-Octyldodecanol | 0.2 |
| Preservatives | q.s. |
| Panthenol | 0.5 |
| Polysorbate-21 (Tween 21 from Croda) | 0.5 |
| Glycerol | 5 |
| Hydroxyethylcellulose | 2 |
| Sodium hyaluronate | 0.1 |
| Acrylamide/sodium 2-acrylamido-2- | 0.6 |
| methylpropanesulphonate copolymer at 40% | |
| as an inverse emulsion in a polysorbate | |
| 80/isohexadecane mixture (Simulgel 600 | |
| from Seppic) | |
| Ethanol | 8 |
| Water | q.s. for 100 |

### Example 5

### Eyeliner

| | Amount |
|---|---|
| Ingredient | (% by weight) |
| L-Arginine | 0.05 |
| Citric acid | 0.05 |
| Madecassoside | 0.3 |
| Preservatives | q.s. |
| Mica/titanium oxide/black iron | 5 |
| oxide/cochineal carmine (Cloisonne Nu | |
| Antique Red 424 CB by Engelhard) | |
| 1,2-Pentanediol | 6.7 |
| Polysorbate-21 (Tween 21 from Croda) | 0.5 |
| Acrylic and styrene/acrylic copolymers as | 55.6 |
| a 40% aqueous emulsion in a | |
| water/butylene glycol/sodium lauryl ether | |
| sulphate mixture (Syntran 5760 from | |
| Interpolymer) | |
| Crosslinked acrylic acid/ethyl acrylate | 5.9 |
| copolymer as a 28% aqueous dispersion | |
| (Aculyn 33 from Röhm & Haas) | |
| Ethanol | 5.57 |
| Water | q.s. for 100 |

### Example 6

### Waterproof Mascara

| | Amount |
|---|---|
| Ingredient | (% by weight) |
| L-Arginine | 0.05 |
| Citric acid | 0.05 |
| Madecassoside | 0.3 |
| Hydroxystearoyl stearate of C₁₈-C₃₈ fatty | 1 |
| alcohols (Kester Wax K82P from Koster | |
| Keunen) | |
| Carnauba wax | 4.7 |
| Beeswax | 7.3 |
| Candelilla wax | 0.1 |
| Paraffin wax | 2.8 |
| Rice bran wax | 2.8 |
| Microdispersion of carnauba wax with 10% | 7 |
| ethanol (Mexoryl SAP from Chimex) | |
| Black iron oxide | 4.2 |
| Preservatives | q.s. |
| Ethylenediamine/stearyl dimer dilinoleate | 1 |
| copolymer (Uniclear 100 VG from Arizona | |
| Chemical) | |
| Poly(vinyl laurate) (Mexomere PP from | 2.2 |
| Chimex) | |
| Vinyl acetate/allyl stearate (65/35) | 3.3 |
| copolymer (Mexomere PQ from Chimex) | |
| Vinylpyrrolidone/eicosene copolymer | 2 |
| (Antaron V220 from ISP) | |
| Talc | 1 |
| Distearyldimethylammonium-modified | 5.8 |
| hectorite (Bentone 38 VCG from Elementis) | |
| Panthenol | 0.01 |
| Polysorbate-21 (Tween 21 from Croda) | 0.5 |
| Ethanol | 2 |
| Propylene carbonate | 1.9 |
| Isododecane | q.s. for 100 |

### Example 7

### Eyebrow Gel

| | Amount |
|---|---|
| Ingredient | (% by weight) |
| Sodium hydroxide | 0.6 |
| L-Arginine | 0.05 |
| Sorbitol as a 70% aqueous solution | 3 |
| Citric acid | 0.05 |
| Madecassoside | 0.3 |
| Guar gum, 2-hydroxypropyl ether | 0.2 |
| 1,2-Octanediol | 0.5 |
| Polysorbate-21 (Tween 21 from Croda) | 0.5 |
| Water | 4 |
| Crosslinked carboxyvinyl homopolymer | 1.5 |
| synthesized in the ethyl | |
| acetate/cyclohexane mixture (Carbopol | |
| Ultrez 10 Polymer from Lubrizol) | |
| Butyl acrylate/acrylic acid/methacrylic | 20 |
| acid branched block polymer in water | |
| (Fixate G100L Polymer from Lubrizol) | |
| Ethanol | 10 |
| Water | q.s. for 100 |

### Example 8

### Waterproof Mascara

| | Amount |
|---|---|
| Ingredient | (% by weight) |
| L-Arginine | 0.05 |
| Citric acid | 0.05 |
| Madecassoside | 0.3 |
| Hydroxystearoyl stearate of C₁₈-C₃₈ fatty | 1 |
| alcohols (Kester Wax K82P from Koster | |
| Keunen) | |
| Carnauba wax | 4.7 |
| Beeswax | 7.3 |
| Candelilla wax | 0.1 |
| Paraffin wax | 2.8 |
| Rice bran wax | 2.8 |
| Microdispersion of carnauba wax with 10% | 7 |
| ethanol (Mexoryl SAP from Chimex) | |
| Black iron oxide | 4.2 |
| Preservatives | q.s. |
| Ethylenediamine/stearyl dimer dilinoleate | 1 |
| copolymer (Uniclear 100 VG from Arizona | |
| Chemical) | |
| Poly(vinyl laurate) (Mexomere PP from | 2.2 |
| Chimex) | |
| Vinyl acetate/allyl stearate (65/35) | 3.3 |
| copolymer (Mexomere PQ from Chimex) | |
| Vinylpyrrolidone/eicosene copolymer | 2 |
| (Antaron V220 from ISP) | |
| Talc | 1 |
| Distearyldimethylammonium-modified | 5.8 |
| hectorite (Bentone 38 VCG from Elementis) | |
| Panthenol | 0.01 |
| Ethanol | 2 |
| Propylene carbonate | 1.9 |
| Isododecane | q.s. for 100 |

### Other Implementational Example of the Method According to the Invention

According to another implementational example, use was made of a vibrating applicator, either alone or in combination with a conventional mascara composition, that is to say not comprising an active principle known for properties of rendering the eyelash more attractive.

The test was carried out on a panel of 30 users, who proceeded as follows:
a) In the morning, they caused a vibrating applicator to engage with their eyelashes (30 strokes) without application of mascara. This simulated application was followed by the application, by means of a nonvibrating applicator, of a conventional mascara composition (not comprising the active principles of the composition according to the invention); and
b) in the evening, they again simulated the application of mascara using a vibrating applicator (30 strokes).

After carrying out the above routine for 4 weeks, a significant improvement in the surface evenness, in the sheen, in the suppleness and in the density of the eyelashes and in the general state of the eyelashes was observed. After 8 weeks, the improvement was also significant for the length of the eyelashes and for the thickness of the eyelashes.

1. A cosmetic composition comprising, in a physiologically acceptable medium:
a) at least one compound selected from the group consisting of arginines, their organic or inorganic acid salts, and their derivatives;
b) at least one compound of formula (I): 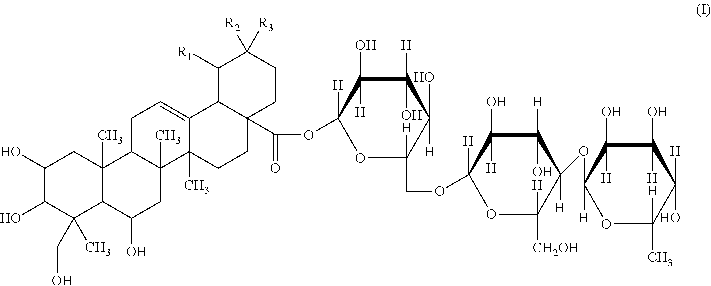
wherein:
R₁=H or —CH₃;
R₂=H or —CH₃;
R₃=—CH₃; and
R₁ and R₂ do not simultaneously equal H,
or a Centella asiatica extract comprising a compound, or a mixture of compounds, of formula (I); and
c) at least one compound of formula (II): 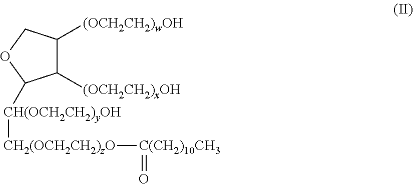
wherein w+x+y+z has a mean value of 4.

2. The composition of claim 1, wherein the compound of formula (I) is at least one selected from the group consisting of madecassoside and terminoloside.

3. The composition of claim 2, wherein the compound of formula (I) is a mixture of madecassoside and terminoloside.

4. The composition of claim 3, wherein the mixture of madecassoside and terminoloside comprises a madecassoside content ranging from 30 to 70% by weight, with respect to the total weight of the mixture.

5. The composition of claim 3, comprising the Centella asiatica extract comprising more than 95% by weight of the mixture of madecassoside and terminoloside, with respect to the weight of the extract.

6. The composition of claim 1, wherein the compound of formula (I) is present in an amount ranging from 0.01 to 5% by weight, with respect to the total weight of the composition.

7. The composition of claim 1, wherein the arginines are selected from the group consisting of D-arginine, D,L-arginine and L-arginine.

8. The composition of claim 1, wherein the arginine salts are selected from the group consisting of hydrochlorides, glutamates, butyrates and glycolates.

9. The composition of claim 1, wherein L-arginine derivatives are selected from the group consisting of C₁-C₄ alkyl esters of L-arginine and L-arginine oligomers comprising from 7 to 15 L-arginine units.

10. The composition of claim 1, wherein the at least one compound a) is present in an amount ranging from 0.01% to 5% of the total weight of the composition.

11. The composition of claim 1, further comprising at least one compound selected from the group consisting of citric acid and its salts.

12. (canceled)

13. A method for cosmetic treatment of human keratinous fibres, comprising applying, to the fibres, the cosmetic composition of claim 1.

14. A combination, comprising:
at least one composition as defined in claim 1; and
at least one applicator for the composition, the applicator comprising a means for smoothing and/or separating eyelashes or eyebrows.

15. The combination of claim 14, wherein the applicator comprises a vibration means and/or a heating means.

16. A combination, comprising:
at least one composition comprising, in a physiologically acceptable medium, at least one active principle capable of inducing and/or stimulating eyelash or eyebrow growth, and
at least one applicator for the composition, the applicator comprising a means for smoothing and/or separating eyelashes or eyebrows, and a vibration means.

17. The combination of claim 16, comprising, in a physiologically acceptable medium:
a) at least one compound selected from the group consisting of arginines, their organic or inorganic acid salts, and their derivatives;
b) at least one compound of formula (I): 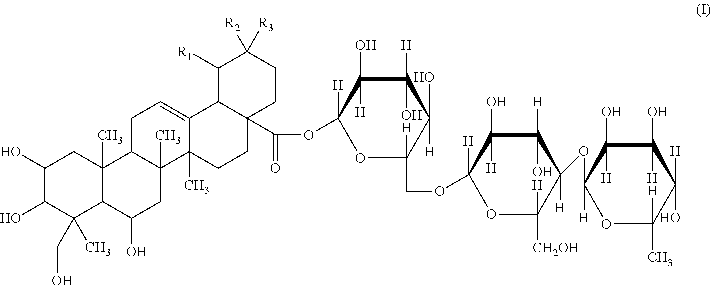
wherein:
R₁=H or —CH₃;
R₂=H or —CH₃;
R₃=—CH₃, and
R₁ and R₂ do not simultaneously being equal H,
or a Centella asiatica extract comprising a compound, or a mixture of compounds, of formula (I); and
c) optionally at least one compound of formula (II): 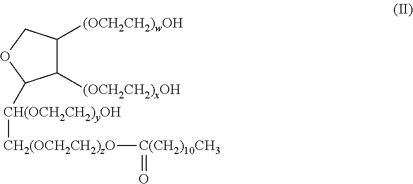
wherein w+x+y+z has a mean value of 4.

18. A method for cosmetic treatment of eyelashes or eyebrows, comprising engaging the eyelashes or eyebrows with an applicator, wherein the applicator is driven with a vibratory movement.

19. The method of claim 18, further comprising, simultaneously with the vibratory movement or separately, applying to the eyelashes or eyebrows, a composition comprising, in a physiologically acceptable medium:
a) at least one compound selected from the group consisting of arginines, their organic or inorganic acid salts, and their derivatives;
b) at least one compound of formula (I): 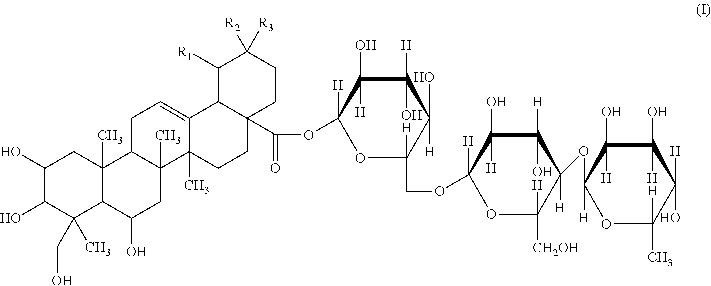
wherein:
R₁=H or —CH₃;
R₂=H or —CH₃;
R₃=—CH₃; and
R₁ and R₂ do not simultaneously equal H,
or a Centella asiatica extract comprising a compound, or a mixture of compounds, of formula (I); and
c) optionally at least one compound of formula (II): 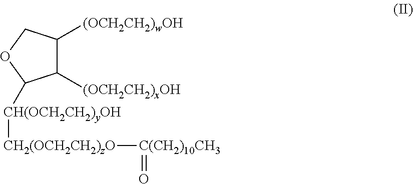
wherein w+x+y+z has a mean value of 4.

20. The method of claim 13, wherein the method induces and/or stimulates the growth of human keratinous fibres and/or slows down their loss and/or increases their density.
